(19) United States
(12) Patent Application Publication
(10) Pub. No.: US 2018/0306651 A1
(43) Pub. Date: Oct. 25, 2018

(54) Apparatus and System for Monitoring an Insulin Sample in a Case

(71) Applicant: beteSTRONGCASES, LLC, Northfield, IL (US)

(72) Inventors: Mark D. Mitchell, Northfield, IL (US); Connor F. Mitchell, Skokie, IL (US)

(73) Assignee: beteSTRONGCASES, LLC

(21) Appl. No.: 15/493,244

(22) Filed: Apr. 21, 2017

(51) Int. Cl. G01K 13/00; A61M 5/142; A61M 5/00

(52) U.S. Cl. CPC G01K 13/00; A61M 5/142; A61M 5/002; A61M 2205/3368; A61M 2205/50; A61M 2205/18; A61M 2205/3331; A61M 2205/584; A61M 2205/3569

(57) ABSTRACT

A monitoring system for monitoring the temperature of a sample of insulin stored in a container. Additionally, a monitoring and detecting system for monitoring a sample that is sensitive to ambient temperature, pressure and/or ultra-violet (UV) radiation or other conditions. The system includes sensor circuits that sense conditions of a sample. A comparator circuit receives input from the sensor circuit indicative of the sensed conditions of the sample. The comparator circuit generates a profile of the sample and indicator modules provide an indication, such as warn or fail for the sample.

### CROSS REFERENCE TO RELATED APPLICATIONS

Not applicable to this application.

### STATEMENT REGARDING FEDERALLY SPONSORED RESEARCH OR DEVELOPMENT

Not applicable to this application.

### BACKGROUND

### Field

Example embodiments in general relate to a detecting and monitoring system for a sample fluid. More particularly, embodiments relate to detecting and monitoring a plurality of ambient conditions of a sample of insulin. Furthermore, an apparatus and system to monitor the temperature of a sample of insulin and/or interior temperature of an insulin storage compartment (including an insulin carrying case, infusion pump case or infusion pump) are disclosed.

### Related Art

Any discussion of the related art throughout the specification should in no way be considered as an admission that such related art is widely known or forms part of common general knowledge in the field. Indeed, the discussion of the prior art is intended to provide some context for the description of the embodiments of the invention.

Typically, a person having diabetes needs to monitor their blood insulin level at prescribed intervals to ensure that the level of insulin in their blood is within an acceptable range. Diabetic patients may utilize a carrying case to hold their insulin vial, trocar, hypodermic needles, test strips and other items needed to treat their blood sugar level.

In the past two decades, insulin infusion pumps have been developed for the treatment of diabetes. Many diabetics prefer to utilize an insulin infusion pump to provide insulin into their body instead of the carrying case. Some advantages to an insulin infusion pump include provision of a more regular introduction of insulin into their body; reduction of the need for daily injections of insulin; and greater mobility since the infusion pump is typically attached via a port and the user does not need to carry a pouch with insulin cartridges, hypodermic needles, test strips, trocar and other medical supplies necessary for injecting insulin. Many infusion pump devices are water-resistant and may be waterproof. Regardless of how insulin is stored, which could be in a carrying pouch, infusion pump case or infusion pump apparatus, it is important that the temperature of the sample does not become too hot or too cold. A concern for insulin users is that the temperature of a sample of insulin may exceed the prescribed temperature (either too hot or too cold) for a period of time and then return to an acceptable temperature. In this instance, the efficacy of the insulin could be compromised and the user would not be aware of the undesirable temperature profile of the insulin.

It would be desirable to have an improved monitoring system that is capable of generating a warning or alert based on sensed conditions of a sample of insulin, either in an insulin carrying case, an infusion pump case and/or an infusion pump apparatus.

### SUMMARY

An example embodiment of the present invention is directed to monitoring the temperature of insulin and/or the temperature of the interior of an insulin storage compartment. The insulin storage compartment, or case, may be, for example, an insulin carrying pouch, an infusion pump case and/or an infusion pump apparatus. As used herein, the insulin carrying pouch is a carrying case that a user typically carries an insulin vial, test strips and other paraphernalia to administer insulin. An infusion pump case is a case for holding an infusion pump. An infusion pump apparatus is an infusion pump without the infusion pump case. One embodiment of the present invention is directed to a monitoring system (“the system”) for monitoring a temperature (or other parameter, such as pressure, UV exposure) of a sample stored in a case. For example, the sample may be insulin contained in an insulin storage case (e.g., a carrying pouch, infusion pump case and/or infusion pump apparatus). The monitoring system includes a case structure adapted to contain a sample of insulin, which may be stored in a vial or other insulin-containing vessel. A temperature sensor is disposed in the interior of the case. The temperature sensor senses a temperature of the interior of the case and/or the temperature of the insulin sample. A comparator is disposed in the interior of the case that receives input from the temperature sensor indicative of the temperature of the interior of the case. The comparator generates an output signal as a function of the sensed temperature of the interior of the case. An indicator is disposed on the case such that at least a portion of the indicator is visible from the exterior of the case, each indicator operatively coupled to the comparator. The indicator provides an indication of the temperature of the interior of the case based on the output signal generated by the comparator of the sample.

Another embodiment of the present invention is directed to the system, wherein the comparator circuit generates a profile of the sample based on one or more parameters. The profile reflects the exposure of the sample to various temperature, pressure and/or UV exposure conditions.

Yet another embodiment is the system, which includes additional sensors, sense ultra-violet radiation exposure of the sample.

Yet another embodiment of the present invention is the system, wherein the sensor senses ambient pressure conditions of the sample.

Yet another embodiment is the system, that includes one or more indicator modules that are each associated with a particular sensed parameter of the sample.

Yet another embodiment is the system that also includes a bi-directional communication link adapted to transmit a representation of the profile, such as a temperature profile, of the sample.

Yet another embodiment is the system, in which the bi-directional communication link is a wireless link.

Yet another embodiment is the system, in which the sensor senses a plurality of conditions.

Yet another embodiment is the system, in which the plurality of conditions include: time, temperature, pressure, and ultra-violet radiation.

Yet another embodiment is the system, in which the comparator generates a warning indication based on the profile of the sample, and the indicator modules are adapted to provide an indication representative of the warning indication.

Yet another embodiment is a method to accomplish the detecting and monitoring of a fluid sample, such as a sample of insulin. The method may comprise a series of steps, or algorithm, or computer program code that may be stored on a non-transitory computer readable medium. The steps may be stored on non-volatile memory, SM card, thumb drive, memory stick, cache, EEPROM, RAM, ROM, DRAM or other suitable electronic memory.

Other embodiments of the present invention include the methods described above but implemented using apparatus or programmed as computer code to be executed by one or more processors operating in conjunction with one or more electronic storage media.

There has thus been outlined, rather broadly, some of the embodiments of the monitoring system in order that the detailed description thereof may be better understood, and in order that the present contribution to the art may be better appreciated. There are additional embodiments of the monitoring system that will be described hereinafter and that will form the subject matter of the claims appended hereto. In this respect, before explaining at least one embodiment of the monitoring system in detail, it is to be understood that the monitoring system is not limited in its application to the details of construction or to the arrangements of the components set forth in the following description or illustrated in the drawings. The monitoring system is capable of other embodiments and of being practiced and carried out in various ways. Also, it is to be understood that the phraseology and terminology employed herein are for the purpose of the description and should not be regarded as limiting.

### BRIEF DESCRIPTION OF THE DRAWINGS

Example embodiments will become more fully understood from the detailed description given herein below and the accompanying drawings, wherein like elements are represented by like reference characters, which are given by way of illustration only and thus are not limitative of the example embodiments herein.

FIG. 1 is a perspective view of a carrying pouch for carrying insulin.

FIG. 2 is a perspective view of an infusion pump case for insulin.

FIG. 3 is a perspective view of an insulin pump apparatus that may or may not be disposed inside an infusion pump case.

FIG. 4 is a diagram of a sensing and monitoring apparatus in accordance with one embodiment.

FIG. 5 is a diagram of a sensing and monitoring apparatus in accordance with another embodiment.

FIG. 6 is a diagram of a sensing and monitoring system in accordance with yet another embodiment.

FIG. 7 is a diagram of a sensing and monitoring system in accordance with yet another embodiment.

FIG. 8 is a series of steps to sense temperature of a sample.

FIG. 9 is a series of steps to sense a plurality of parameters of a sample.

FIG. 10 shows a diagram of time and temperature conditions according to an embodiment.

FIG. 11 shows a diagram of time and pressure conditions according to an embodiment.

FIG. 12 shows a diagram of time and ultra-violet radiation conditions according to an embodiment.

FIG. 13 shows an example representation of a display according to an embodiment.

FIG. 14 shows an example of an insulin storage case with one or more temperature sensors according to an embodiment.

FIG. 15 shows an example of an insulin pump case with temperature sensors according to an embodiment.

### DETAILED DESCRIPTION

As used in this application, the terms “component” and “system” are intended to refer to a computer-related entity, either hardware, a combination of hardware and software, software, or software in execution. For example, a component may be, but is not limited to being, a process running on a processor, a processor, an object, an executable, a thread of execution, a program, and/or a computer. By way of illustration, both an application running on a server and the server can be a component. One or more components may reside within a process and/or thread of execution and a component may be localized on one computer and/or distributed between two or more computers.

Embodiments of the present invention provide an insulin case, which may include an infusion pump case, that has temperature sensing circuitry to sense and monitor the internal temperature of the insulin case. When the internal temperature of the case exceeds a predetermined threshold, such as 80 degrees Fahrenheit, a buzzer (or audible alert), LED (light emitting diode), or other indicator is activated. This indication provides a user, with an alert that the internal temperature of the insulin case has exceeded the predetermined threshold.

An example monitoring system generally comprises sensing circuits, a comparator circuit and indicator modules. A sample used for treating a condition, such as insulin for treating diabetes, may be susceptible to changes in ambient conditions, such as, for example, temperature, pressure and/or ultra-violet exposure. Other ambient conditions may also deleteriously affect the efficacy of a therapy. As described and claimed herein, it is contemplated that such ambient conditions can be detected and/or monitored such that a user, patient or other party will have a profile of conditions that a therapy, such as insulin, has been exposed.

Specifically, a therapy, such as insulin, may be exposed to an undesirable temperature for a short period of time. It is useful to a patient to have insight into the temperature profile of the therapy, even if the temperature of the therapy returns to a lower, “acceptable” level following an elevated temperature period. This profile technique may be used to develop a profile of any suitable parameter (i.e., temperature, pressure, UV exposure, etc.) for any suitable therapy to provide an indication of suitability of the sample based on what conditions the sample experienced.

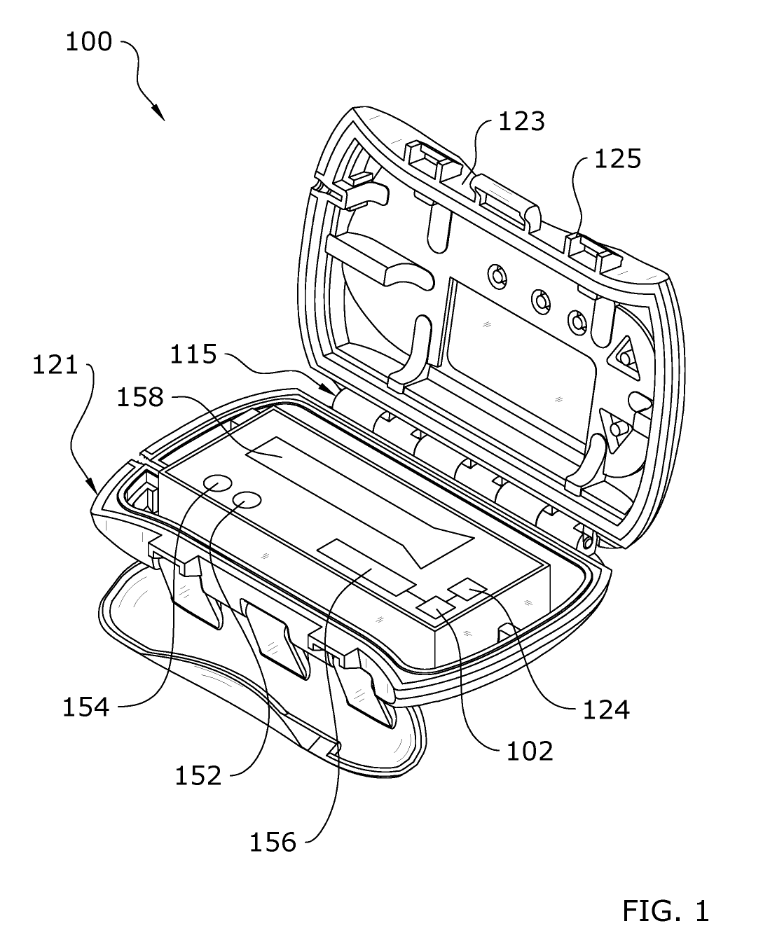

FIG. 1 is a perspective view of an insulin storage case 100 for carrying insulin. The insulin storage case 100 includes a lower, or bottom, member 121 and an upper member 123. The bottom member 121 and upper member 123 are hinged together with a hinge portion 115. Attachment portion 125 of upper member 123 is used to attach upper member 123 to lower member 121 such that the insulin storage case 100 may be opened. The insulin storage case 100 portions could also be attached via a zipper, clips or other mechanism for affixing the portions of the insulin storage case 100.

Lower member 121 includes a surface for storing diabetes treatment components and sensing components. Insulin storage case 100 includes one or more sensors, shown as single temperature sensor 102. The insulin storage case 100 is adapted to hold a testing device, which may be, for example, a skin puncture device 158, and an insulin vial 156, which stores a sample of insulin that is to be injected into a patient.

The temperature sensor 102 is used to sense the temperature inside the insulin storage case 100 and may also be disposed to specifically sense the temperature of insulin sample vial 156. The sensor 102 is operatively coupled to comparator circuit 124.

The sensor 102 may be disposed such that it senses the temperature inside the insulin storage case 100 or the temperature of the insulin vial 156. It may be desirable to sense the temperature of the insulin sample in vial 156 since the temperature of the insulin sample in vial 156 may vary with the volume of insulin in the vial 156.

The comparator circuit 124 compares the sensed temperature sensed by the sensor 102 to a pre-determined threshold to determine whether or not the sensed temperature inside the insulin storage case 100, or of the insulin vial 156, exceeds a pre-determined threshold. Typically the threshold temperature is set to 80 degrees Fahrenheit.

Indicator 134 is disposed on the insulin storage case 100 such that at least a portion of the indicator 134 is visible from outside the insulin storage case 100, such that a user can see the status of the insulin. The indicator 134 may display an illuminated light, provide a buzzer alert, audible sound, provide a multi-colored illuminated alert or provide a combination of illumination of colors and/or sounds. The audible indication may be a tone that is a function of the output signal generated by the comparator 124. Compartments 152 and 154 may be used to contain test strips, hypodermic needle(s), or other items useful to test a user's blood.

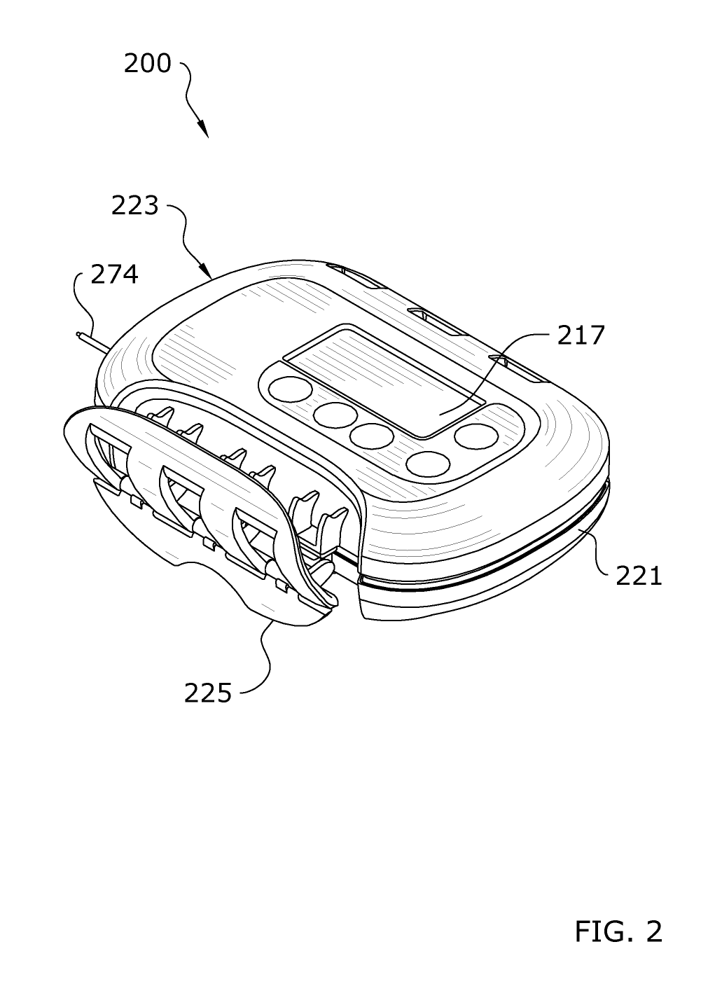

FIG. 2 is a perspective view of an infusion pump case 200 for insulin. The infusion pump case 200 is suitable for holding an infusion pump apparatus. The infusion pump case 200 includes upper portion 223 and lower portion 221. Attachment hinge 225 is used to attach upper portion 223 to lower portion 221. Infusion pump case 200 has tube 274 to provide a conduit for insulin from the infusion pump to the patient. Display screen 217 is used to provide information to a user regarding the contents of infusion pump case 200. This information may relate to a profile of the insulin contained in the pump case 200. The display 217 may be an LCD, plasma screen, or other suitable display device.

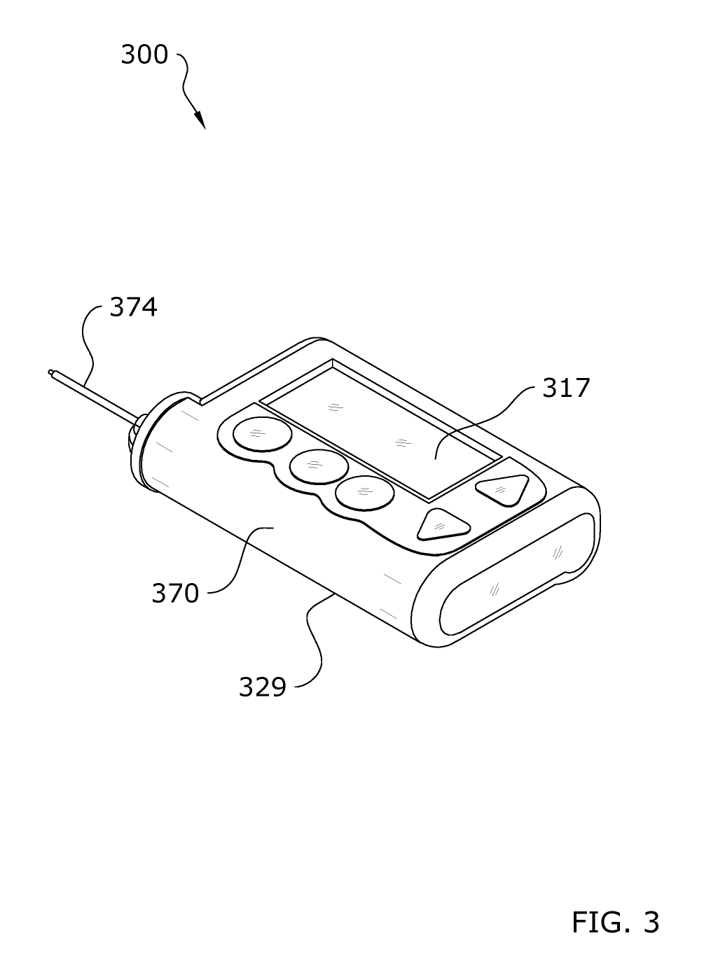

FIG. 3 is a perspective view of an insulin pump apparatus 300 that may or may not be disposed inside an infusion pump case (as described herein). The infusion pump apparatus 300 has a body section 329. The body section 329 includes an insulin pump 370 to pump insulin into the patient via conduit tubing 374. Display module 317 is, for example, an LED (light emitting diode), plasma screen, LCD (liquid crystal display) or other suitable display to provide a visual indication of the state of insulin contained in insulin pump 370.

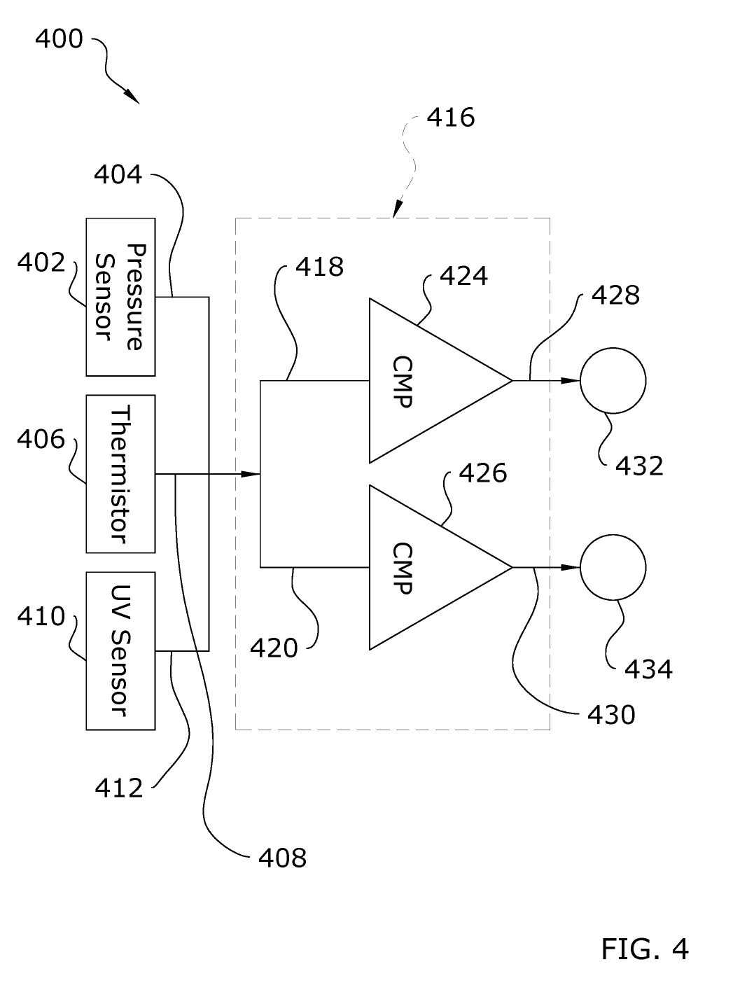

FIG. 4 is a diagram of a sensing and monitoring apparatus in accordance with one embodiment. The apparatus 400 includes sensing circuits, a comparator circuit and indicator modules.

Sensing circuits 402, 406, 410 are shown as a pressure sensor, thermistor and ultra-violet sensor, respectively. A pressure sensor 402 is used to sense ambient pressure. A thermistor 406 is used to sense ambient temperature. A ultra-violet (UV) radiation sensor 410 is used to sense exposure to UV radiation. The sensing circuits 402, 406 and 410 are operatively coupled to comparator circuit 416 via wires 404, 408 and 412, respectively.

Comparator circuit 416 receives input from the sensing circuits 402, 406, 410. The comparator circuit 416, as shown in FIG. 4 includes two comparators 424, 426, that receive input via wires 418 and 420, respectively. Comparators 424, 426 may be, for example, op amps, or other suitable circuitry that outputs a signal based on a comparison of a sensed condition, from the sensors, to a threshold. The comparator circuit 416 produces two outputs to indicator modules 432, 434, via wires 428 and 430, respectively. The indicator modules 432, 434 may be light emitting diodes (LEDs) that produce a color indication based on the output signal from the comparator circuit 416. The use of two indicator modules 432, 434 enables an output indication that may be a single color (red) or a second color (yellow).

Alternatively, the system of FIG. 4 could be implemented with a single indicator module that provides a single indication. The use of multiple indicators may also be used so that each indicator is associated with a particular parameter and/or threshold level.

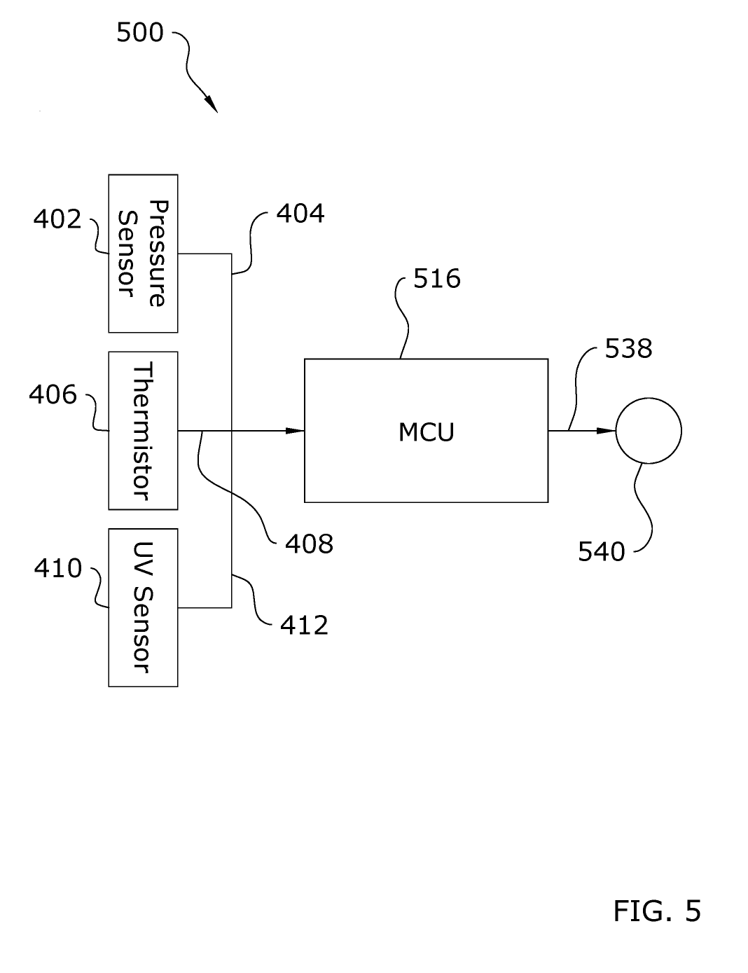

FIG. 5 is a diagram of a sensing and monitoring apparatus in accordance with another embodiment. As shown in FIG. 5, system 500 includes sensing circuits 402, 406, 410 are shown as a pressure sensor, thermistor and ultra-violet sensor, respectively. A pressure sensor 402 is used to sense ambient pressure. A thermistor 406 is used to sense ambient temperature. An ultra-violet (UV) radiation sensor 410 is used to sense exposure to UV radiation.

The sensing circuits 402, 406 and 410 are operatively coupled to comparator circuit 516 via wires 404, 408 and 412, respectively. There may be more or fewer sensing circuits than shown in FIG. 4.

Comparator circuit 516 receives input from the sensing circuits. The comparator circuit 516, as shown in FIG. 5, is used to receive the inputs from the sensing circuits 402, 406, 410 and generate a signal indicative of the sensed parameters. The comparator circuit 516 produces an output signal to indicator module 540 via wire 538. The indicator module 540 may be light emitting diode (LED) that produces a color indication based on the output signal from the comparator circuit 516. The indicator 540 may be a single LED with multiple colors such that the LED may be illuminated as entirely one color (e.g., green), or illuminated as a blend of colors (e.g., green, red and yellow). The number of colors and pattern of an indication using a single LED may be predetermined such that, for example, an all green display means that the insulin sample has not been degraded. An all red display could mean that the sample has been degraded beyond efficacy and a blend of green and red could be a sliding scale of degradation.

The output from the comparator circuit 516 may include information indicative of a sensed condition over time of multiple sensed parameters. This profile generated by the comparator circuit 516 is useful to provide information of conditions that may have been previously present; but are not currently present, which have a bearing or affect on the therapy. For example, it is possible that a sample of a therapy, such as insulin, may have been exposed to elevated temperatures that exceed the permissible temperature; but, the sample was then cooled to a permissible temperature. It is important for a patient or user of the sample to be aware of the higher temperature exposure of the sample that previously occurred.

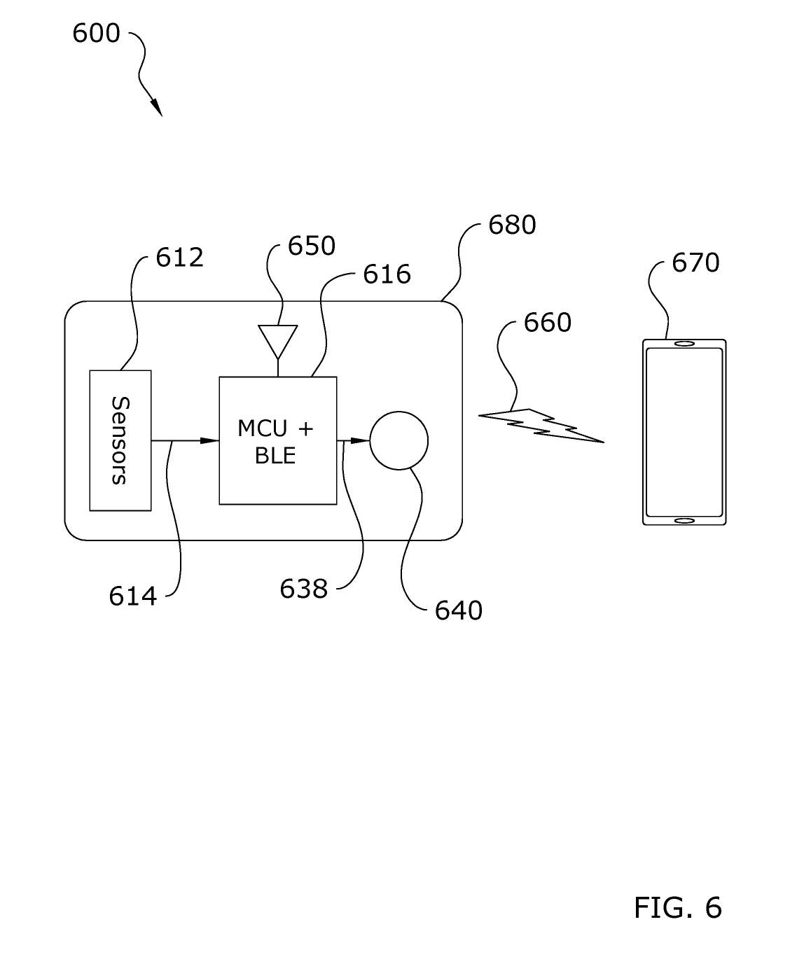

FIG. 6 is a diagram of a sensing and monitoring system 600 in accordance with yet another embodiment. As shown in FIG. 6, system 600 includes a detecting unit 680, wireless communication 660 and user device 670.

The detection unit 680 includes sensing circuit 612, comparator circuit 616, display module 640 and antenna 650. The detection unit 680 is adapted to communicate with a user device 670 (having a display screen, or other output mechanism), via wireless transmission 660.

The sensing circuit 612 performs sensing of ambient conditions such as temperature, pressure, UV exposure, acoustic wave sensing, active sensing and passive sensing. The sensing circuit 612 is operatively coupled to comparator circuit 616 via wire 614.

Comparator circuit 616 receives input from the sensing circuit 612. The comparator circuit 616 generates a signal indicative of the sensed parameters. The comparator circuit 616 produces an output signal to indicator module 640 via wire 638. The indicator module 640 may be light emitting diode (LED) that produces a color indication based on the output signal from the comparator circuit 616. The output from the comparator circuit 616 may include information indicative of a sensed condition over time of multiple sensed parameters. This profile generated by the comparator circuit 616 is useful to provide information of conditions that may have been previously present; but are not currently present, which have a bearing or affect on the therapy.

Comparator circuit 616 has a transmit antenna 650, which may communicate, via wireless communication 660, such as a wireless network, Ethernet or other suitable electronic communication technique, with a user device 670. The network 660 is, for example, any combination of linked computers, or processing devices, adapted to transfer (transmit and/or receive) and process data. The network 660 may include wireless and wired transmission capabilities. The network 660 may be a private Internet Protocol (IP) network, as well as a public IP network, such as the Internet that can utilize World Wide Web (www) browsing functionality. Alternatively, the network 660 may be an Ethernet network, or any two or more operatively coupled processing devices that can share information. User device 670 may be any suitable network enabled device with sufficient memory, processing capability and display functionality (such as a touch screen, LCD, plasma or other display) to receive and display a representation of profile data generated by comparator circuit 616. The user device 670 may be for example, a smart phone, tablet, personal computer or any other Internet enabled device. The profile information may be transmitted from the device 680 to the user device 670.

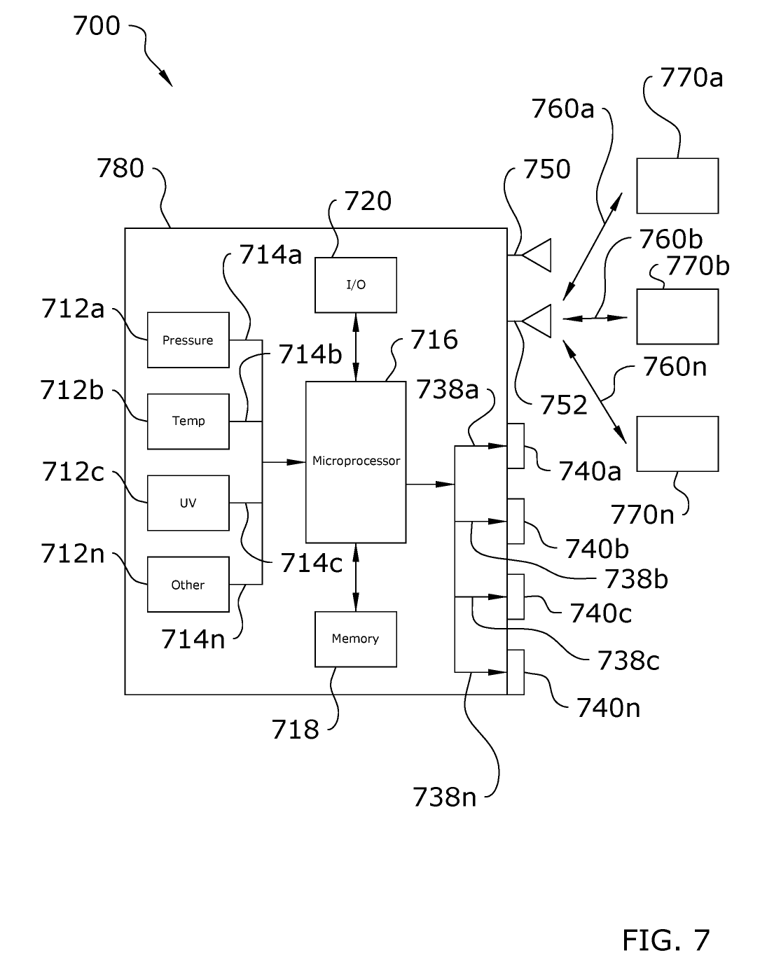

FIG. 7 is a diagram of a sensing and monitoring system 700 in accordance with yet another embodiment. As shown in FIG. 7, system 700 includes a detecting/monitoring unit 780, wireless communication 760 and user devices 770(a) . . . (n) (where “n” is any suitable number).

The unit 780 includes sensing circuits 712(a) . . . (n) (where “n” is any suitable number), microprocessor 716, memory 718, input/output module 720, display modules 740(a) . . . (n) (where “n” is any suitable number) and antennae 750, 752. Two antennae (750, 752) are shown; however, any number of antennae that facilitate transmission/reception to unit 780 may be used. The unit 780 is adapted to communicate with a user devices 770(a) . . . (n) (having a display screen, or other output mechanism), via wireless transmission paths 760(a) . . . (n) (where “n” is any suitable number).

The sensing circuits 712(a) . . . (n) (where “n” is any suitable number) perform sensing of ambient conditions such as pressure (712(a)), temperature (712(b)), UV exposure (712(c)), acoustic wave sensing, active sensing and passive sensing (other 712(n)). The sensing circuits 712(a) . . . (n) are operatively coupled to microprocessor 716 via associated wires 714(a) . . . (n)(where “n” is any suitable number).

Microprocessor 716 receives input from the sensing circuits (generally 712). The microprocessor 716 is operatively coupled to memory 718 and input/output (I/O) module 720. Memory module 718 may be, for example, volatile or non-volatile memory, RAM, ROM, DRAM, EEPROM or other suitable electronic memory. The I/O module 720 may be, for example, instructions encoded on electronic memory to facilitate operation of the microprocessor.

The microprocessor 716, with interaction with memory 718 and I/O module 720, generates a signal indicative of the sensed parameters or sensed conditions. The microprocessor 716 produces an output signal to one or more of indicator modules 740(a) . . . (n) (where “n” is any suitable number) via associated wires 738(a) . . . (n). Each of the indicator modules (generally 740) may be light emitting diode (LED) that produces a color indication based on the output signal from the microprocessor 716. The output from the microprocessor 716 may include information indicative of a sensed condition over time of multiple sensed parameters. This profile generated by the microprocessor 716 is useful to provide information of conditions that may have been previously present; but are not currently present, which have a bearing or affect on the therapy.

Detecting/monitoring unit 780 has transmit antennae 750, 752, which may communicate, via wireless communication 760 (a) . . . (n) (where “n” is any suitable number), such as a wireless network, Ethernet or other suitable electronic communication technique, with one or more user devices 770(a) . . . (n) (where “n” is any suitable number).

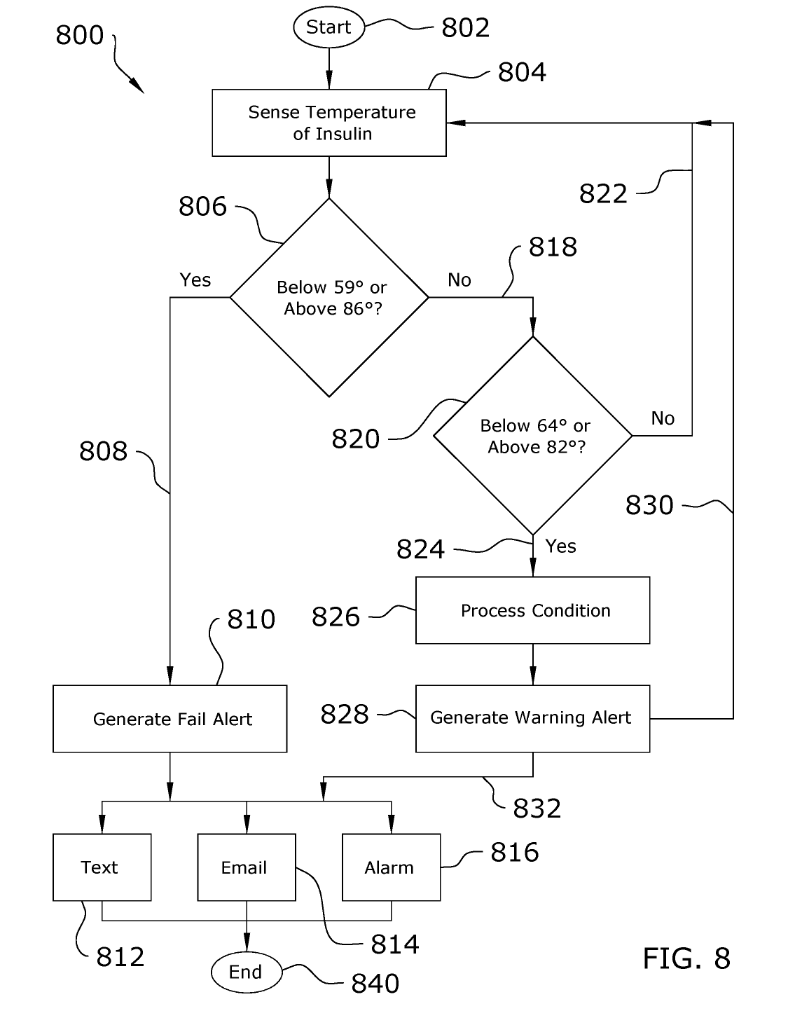

The network 760 is, for example, any combination of linked computers, or processing devices, adapted to transfer (transmit and/or receive) and process data. The network 760 may include wireless and wired transmission capabilities. The network 760 may be a private Internet Protocol (IP) network, as well as a public IP network, such as the Internet that can utilize World Wide Web (www) browsing functionality. Alternatively, the network 760 may be an Ethernet network, or any two or more operatively coupled processing devices that can share information. User device (generally 770) may be any suitable network enabled device with sufficient memory, processing capability and display functionality (such as a touch screen, LCD, plasma or other display) to receive and display a representation of profile data generated by unit 780. The user device 770 may be for example, a smart phone, tablet, personal computer or any other Internet enable device. The profile information may be transmitted from the unit 780 to the user device 770. FIG. 8 is a series of steps 800 to sense temperature of a sample. The steps 800 may be stored in a non-transitory computer readable medium such as EEPROM, DRAM, ROM, or RAM, or other suitable electronic medium. The series of steps start, as shown in step 802. The temperature of the sample, such as insulin, is performed as shown in step 804. A determination is made, as shown in step 806, whether the temperature of the sample is below 59 degrees Fahrenheit or above 86 degrees Fahrenheit. If so, “yes” line 808 shows that a “Fail” alert is generated, as shown in step 810.

The fail alert may be displayed as a text, step 812, e-mail, step 814 and/or alarm, step 816. Step 840 shows that the series of steps ends.

If the determination in step 806 is that the temperature of the sample is not below 59 degrees Fahrenheit or above 86 degrees Fahrenheit, “no” line 818 shows that a determination is made whether the sample temperature is below 64 degrees Fahrenheit or above 82 degrees Fahrenheit, as shown in step 820. (These temperature ranges are based on optimum temperatures for a sample of insulin. Other temperature ranges may be used for other types of samples.)

If the result of the determination (step 820) is that the sample temperature is not below 64 degrees Fahrenheit or above 82 degrees Fahrenheit “no” line 822 shows that the process then continues to sense the sample (step 804).

If the result of the determination (step 820) is that the sample temperature is below 64 degrees Fahrenheit or above 82 degrees Fahrenheit “yes” line 824 shows that the process then continues to process the condition, as shown in step 826. This step includes identifying a time period that the temperature has been outside the acceptable range. A warning alert is generated, as shown in step 828. Line 832 shows that the alert may be a text alert (step 812), e-mail alert (step 814) and/or an alarm (step 816). Line 830 shows that once a warning alert has been generated, the temperature of the sample is continually sensed (step 804). This continual sensing enables a profile of a parameter (e.g., temperature) to be generated by accumulating sensed data over time. As discussed previously, once an alert, such as either fail (step 810) or warning (step 828) has been generated, end step 840 is reached.

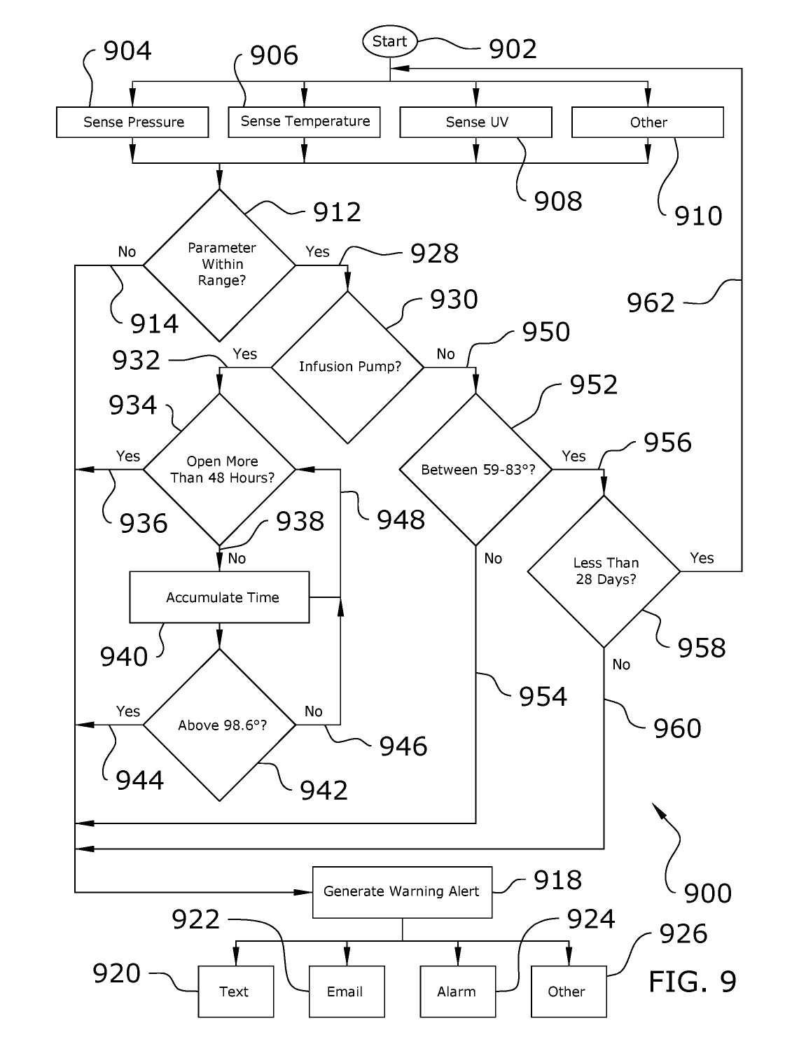

FIG. 9 is a series of steps, or algorithm, to sense a plurality of parameters of a sample. The series of steps 900 may be stored on a non-transitory computer readable medium, such as EEPRPM, ROM, memory stick, thumb drive, cache memory or other suitable electronic storage medium.

The process starts, as shown in step 902. Conditions, or parameters, of a sample: pressure; temperature; UV exposure; and others (e.g., acoustic sensing, active sensing, and/or passive sensing) are sensed as shown in steps 904, 906, 908, and 910, respectively. A determination is made whether each of the sensed conditions are within an acceptable range, as shown in step 912. The conditions/parameters and acceptable ranges that are used in the determination may be programmed based on the sample under consideration. The parameters and ranges may be updated or modified by modifying associated code.

If the sensed parameters are not within an acceptable range for any one of the sensed parameters, “no” line 914 shows that a warning is generated, as shown in step 918. This warning may be provided as a text (920), e-mail (922), alarm (924) or other suitable indication (926).

If the determination, as shown in step 912, results in each of the sensed parameters being within the pre-specified ranges, “yes” line 928 shows that a determination is made whether the sample, insulin in this example, is in an infusion pump, as shown in step 930. If so, “yes” line 932 shows that a determination is made whether the sample has been open for more that 48 hours, as shown in step 934. Insulin in an infusion pump has additional conditions of not being opened for more than 48 hours and not reaching a temperature over 98.6 degrees Fahrenheit. The infusion pump scenario for insulin is an example of a therapy having various additional conditions that affect the efficacy of the therapy. If so, “yes” line 936 leads to warning generation step 918.

If the insulin sample in an infusion pump has not been open for more than 48 hours, “no” line 938 shows that time that the insulin sample has been open is accumulated, as shown in step 940. Line 948 shows that the accumulated open time is then compared to the 48 hour threshold, as shown in step 934.

In addition to accumulating time open, the temperature of the infusion pump insulin is compared to 98.6 degrees Fahrenheit, as shown in step 942. If the temperature of the sample is more than 98.6 degrees Fahrenheit, “yes” line 944 leads to warning generation step 918. If the temperature of the sample is not more than 98.6 degrees Fahrenheit, “no” line 946 leads to determining the duration of time that the sample has been open, as shown in step 934.

If the insulin sample is determined not to be in an infusion pump, as shown in step 930, “no” line 950 shows that a temperature sensing determination is made to determine if the non-infusion pump sample temperature is between 59 and 83 degrees Fahrenheit, as shown in step 952. If not, “no” line 954 shows that warning generation step 918 is reached.

If the non-infusion pump sample temperature is between 59 and 83 degrees Fahrenheit, as shown in step 952 “yes” line 956 shows a determination is made whether or not the sample is less than 28 days old. Typically insulin has reduced efficacy after 28 days, even if not in an infusion pump. If the sample is not less than 28 days old, “no” line 960 shows that warning generation step 918 is reached. If the sample is less than 28 days old, “yes” line 962 leads to the sensing steps 904, 906, 908, 910.

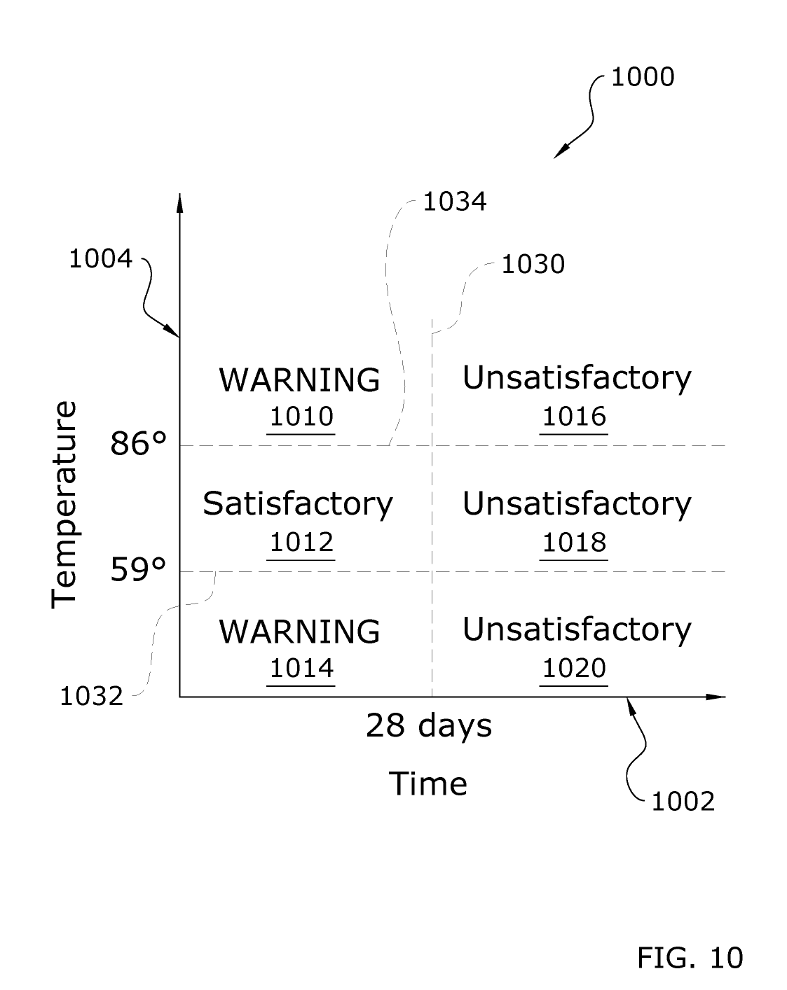

FIG. 10 shows a diagram 1000 of time and temperature conditions according to an embodiment. As shown in the diagram 1000, time is plotted on the x-axis 1002 and temperature is plotted on y-axis 1004. Temperature point at 59 degrees Fahrenheit is shown by line 1032 and temperature point at 86 degrees Fahrenheit is shown by line 1034. A time duration of 28 days is shown by line 1030. The “open” time is time at which the manufacturer's seal is broken and the patient begins to use insulin from that vial or container. There are various areas of the diagram (1014, 1020, 1012, 1032, 1010 and 1016) that result in an associated condition of the therapy. FIG. 10 shows an example for insulin; however, other therapies are also contemplated and can be implemented by adjusting the threshold levels.

The portion of the diagram (1014) indicates that the sample is less than 28 days old and the temperature is below 59 degrees. This is a warning alert condition, since while the sample is less than 28 days old, which is an acceptable condition, the temperature is below 59 degrees, which is a less than optimum condition for insulin.

The portion of the diagram (1020) indicates that the sample is more than 28 days old and the temperature is below 59 degrees, which is an unsatisfactory alert condition. The sample is more than 28 days old, which is an unacceptable condition, and the temperature is below 59 degrees Fahrenheit, which is a less than optimum condition for insulin. Since both the temperature and “open” time of the sample are undesirable, the unsatisfactory alert is generated.

The portion of the diagram (1012) indicates that the sample is less than 28 days old and the temperature is above 59 degrees and below 86 degrees, which is a satisfactory condition. The sample is less than 28 days old, which is an acceptable condition, and the temperature is between 59 and 86 degrees, which is a satisfactory condition for insulin. Since both the temperature and “open” time of the sample are satisfactory, the satisfactory alert is generated.

The portion of the diagram (1018) indicates that the sample is more than 28 days old and the temperature is between 59 and 86 degrees. The sample being more than 28 days old is an unacceptable condition. The temperature is between 59 and 86 degrees, which is a satisfactory condition for insulin. Since the temperature is satisfactory; but the “open” time of the sample is unacceptable, an unsatisfactory alert is generated.

The portion of the diagram (1010) indicates that the sample is less than 28 days old and the temperature is above 86 degrees, which is an unsatisfactory alert condition. The sample is less than 28 days old, which is an acceptable condition. The temperature is above 86 degrees, which is a less than optimum condition for insulin. Since the temperature is less than optimum and “open” time of the sample is satisfactory, the warning alert is generated.

The portion of the diagram (1016) indicates that the sample is more than 28 days old and the temperature is above 86 degrees, which is an unsatisfactory alert condition. The sample is more than 28 days old, which is an unacceptable condition. Since both the temperature and “open” time of the sample are undesirable, the unsatisfactory alert is generated.

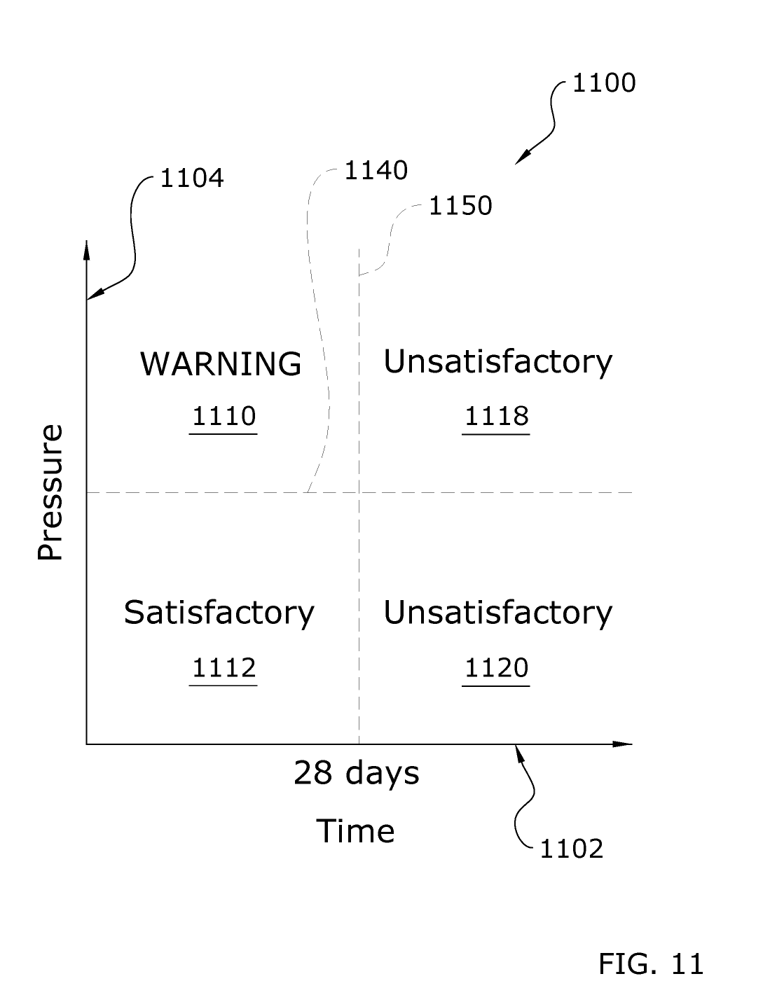

FIG. 11 shows a diagram 1100 of time and pressure conditions. Pressure conditions may be described in terms of “efficacy pressure”, which is a pressure level above which causes the insulin to be less effective. As shown in the diagram 1100, time is plotted on the x-axis 1102 and pressure is plotted on y-axis 1104. Ambient pressure that is a threshold condition for sample efficacy is shown as horizontal line 1140. An “open” time duration of 28 days is shown by vertical line 1150. There are various areas of the diagram (1112, 1120, 1110 and 1118) that result in an associated condition of the therapy.

While FIG. 11 shows an example for insulin, other therapies are also contemplated and can be implemented by adjusting the threshold levels of ambient pressure and “open” time. The threshold levels may be pre-set based on a specific sample and may also be based on a priori information about each sample.

The portion of the diagram (1112) indicates that the sample is less than 28 days old and the pressure is below the efficacy threshold pressure. Since both time and pressure are in an acceptable condition, the sample is satisfactory.

The portion of the diagram (1120) indicates that the sample is more than 28 days old and the pressure is acceptable. Since the “open” time of the sample is undesirable, the unsatisfactory alert is generated.

The portion of the diagram (1110) indicates that the sample is less than 28 days old and the pressure is above the efficacy threshold pressure. Since the time condition is acceptable; but, the pressure is above the efficacy pressure, a warning alert is generated.

The portion of the diagram (1118) indicates that the sample is more than 28 days old and the pressure is above the efficacy pressure. Since both parameters (the “open” time of the sample and the efficacy pressure) are undesirable, the unsatisfactory alert is generated.

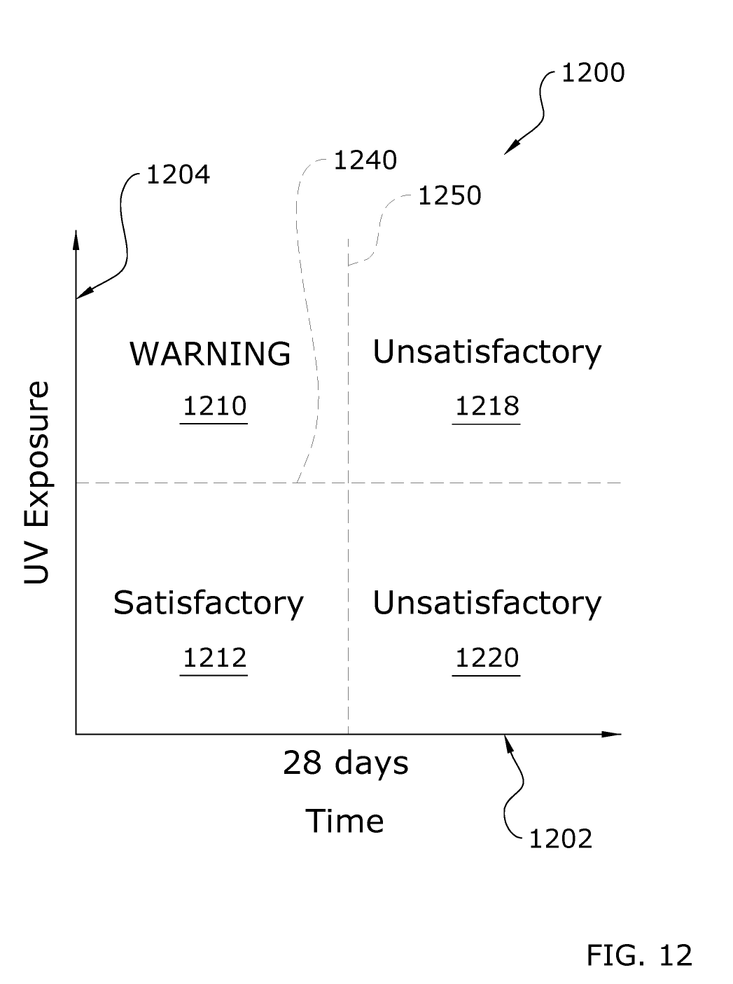

FIG. 12 shows a diagram 1200 of time and ultra-violet radiation conditions according to an embodiment. As shown in the diagram 1200, time is plotted on the x-axis 1202 and UV exposure intensity is plotted on y-axis 1204. UV exposure intensity that is a threshold condition for sample efficacy is shown as horizontal line 1240.

An “open” time duration of 28 days is shown by vertical line 1250. There are various areas of the diagram (1212, 1220, 1210 and 1218) that result in an associated condition of the therapy.

FIG. 12 shows an example for insulin; however, other therapies are also contemplated and can be implemented by adjusting the threshold levels of UV exposure intensity and “open” time. The portion of the diagram (1212) indicates that the sample “open” time is less than 28 days old and the UV exposure intensity is below the efficacy threshold. Since both time and UV exposure intensity are in an acceptable condition, the sample is satisfactory.

The portion of the diagram (1220) indicates that the sample “open” time is more than 28 days and the UV exposure intensity is acceptable. Since the “open” time of the sample is undesirable, the unsatisfactory alert is generated.

The portion of the diagram (1210) indicates that the sample “open” time is less than 28 days and the UV exposure intensity is above the efficacy threshold. Since the time condition is acceptable; but, the UV exposure condition is above the efficacy threshold, a warning alert is generated.

The portion of the diagram (1218) indicates that the sample “open” time is more than 28 days and the UV exposure is above the efficacy threshold. Since both parameters (the “open” time of the sample and the UV exposure) are undesirable, the unsatisfactory alert is generated.

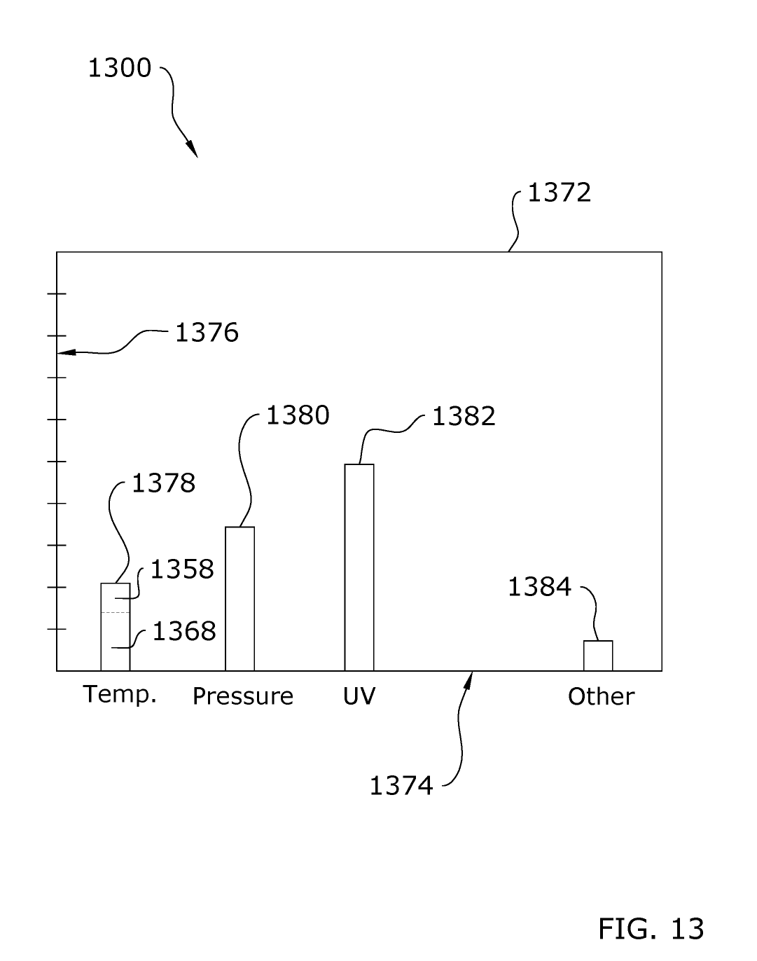

FIG. 13 shows an example representation 1300 of a display according to an embodiment. As shown in FIG. 13, a display 1372 may be used to provide an indication for the efficacy of a sample, such as insulin, or other therapy. The display 1372 may be a touch screen, LCD (liquid crystal display), plasma, or other suitable display to provide a visual indication of the condition of a sample as a function of sensed parameters. The sensed parameters: temperature 1378; pressure 1380; UV 1382; and other sensed parameters 1384 are depicted as a bar graph on horizontal axis 1374, relative to a vertical axis 1376. As part of the bar graph representation (1378, 1380, 1382, 1384) a section of the graph (i.e., 1378) may be used to show warnings (1368) and unsatisfactory indicators (1358).

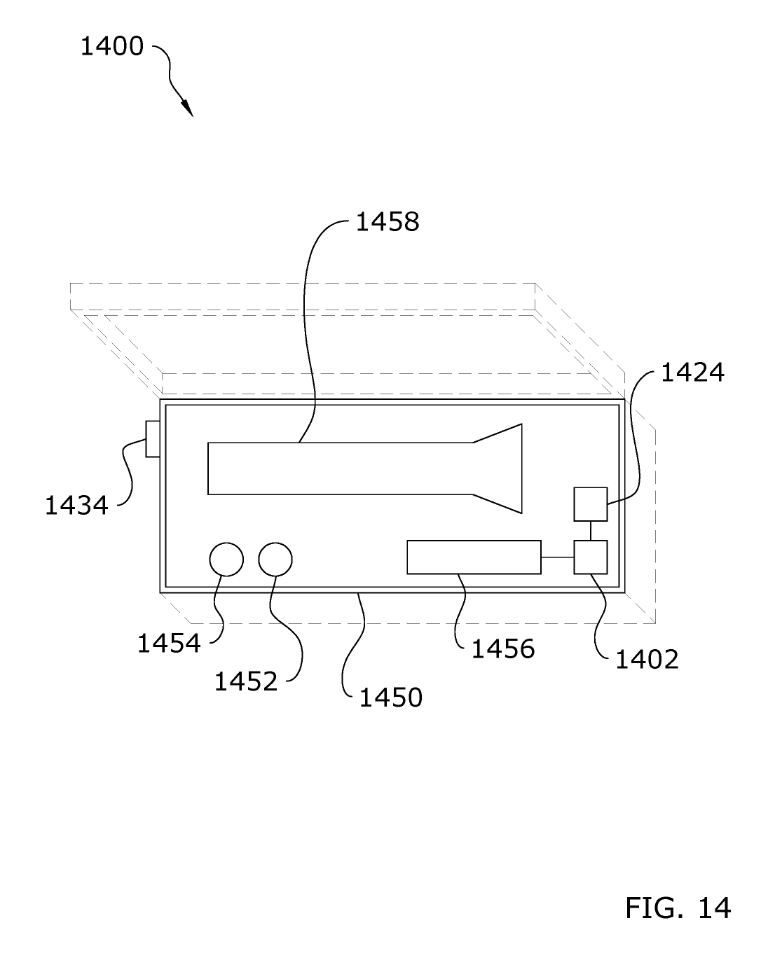

FIG. 14 shows an example 1400 of an insulin storage case with one or more temperature sensors according to an embodiment. As shown in FIG. 14, insulin storage case 1450 includes one or more sensors, shown as single temperature sensor 1402. The insulin storage case 1450 is adapted to hold a testing device, which may be for example a skin puncture device 1458, and insulin vial 1456, which stores a sample of insulin that is to be injected into a patient. The temperature sensor 1402 is used to sense the temperature inside the insulin case 1450 and may also be disposed to specifically sense the temperature of insulin sample vial 1456. The sensor 1402 is operatively coupled to comparator circuit 1424.

The sensor 1402 may be disposed such that it senses the temperature inside the storage case 1450 or the temperature of the insulin vial 1456. It may be desirable to sense the temperature of the insulin sample in vial 1456 since the temperature of the insulin sample in vial 1456 may vary with the volume of insulin in the vial.

The comparator circuit 1424 compares the sensed temperature sensed by the sensor 1402 to a pre-determined threshold to determine whether or not the sensed temperature inside the insulin case 1450, or of the insulin vial 1456, exceeds the pre-determined threshold. Typically the threshold temperature is set to 80 degrees Fahrenheit.

Indicator 1434 is disposed on the case 1450 such that at least a portion of the indicator 1434 is visible from outside the case 1450. The indicator 1434 may display an illuminated light, provide a buzzer alert audible sound, provide a multi-colored illuminated alert or provide a combination of illumination of colors and/or sounds. Compartments 1452 and 1454 may be used to contain test strips, hypodermic needles, or other items useful to test a user's blood.

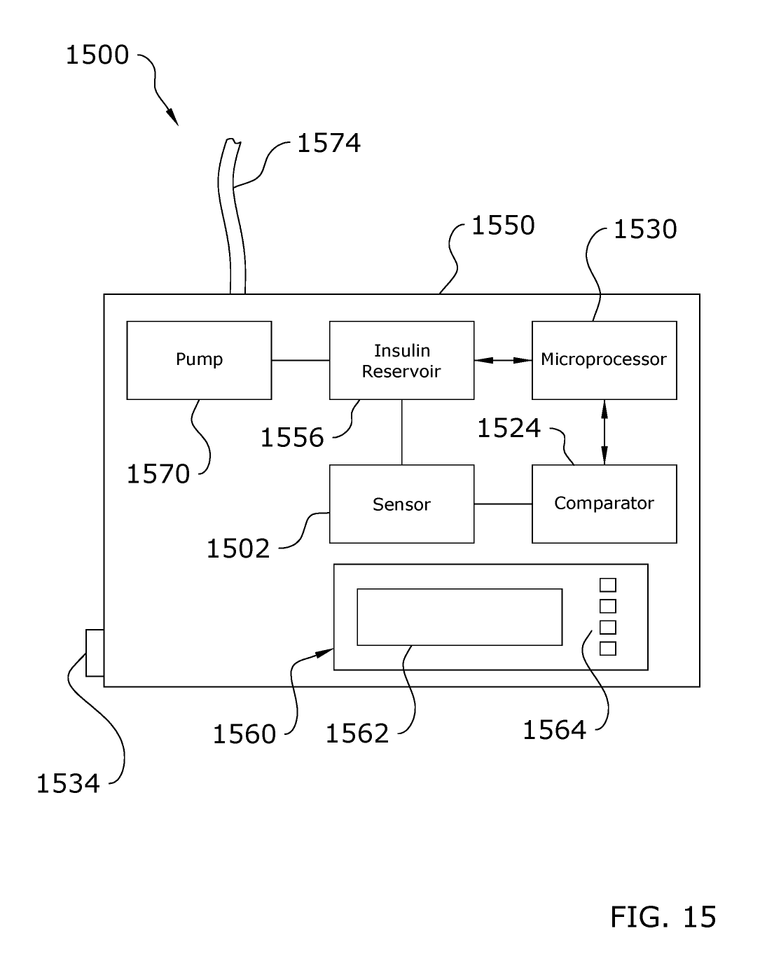

FIG. 15 shows an example 1500 of an insulin infusion pump case 1550 with sensors 1502 according to an embodiment. The infusion pump case 1550 may be water proof, shock-resistant and otherwise adapted to be worn by a user.

The infusion pump case 1550 has an insulin reservoir 1556 for storing a sample of insulin that is to be pumped into a user via pump 1570 and insertion tubing 1574. The insulin reservoir 1556 is operatively coupled to temperature sensor 1502, which can sense either the temperature inside the infusion pump case 1550 or the temperature of insulin reservoir 1556. As stated previously, sensing the temperature of insulin reservoir 1556 may reflect the temperature of the sample inside the reservoir 1556.

Microprocessor 1530 is operatively coupled to comparator 1524, indicator 1534, display module 1560 and reservoir 1556. The microprocessor 1530 controls operation of the infusion pump 1570.

The comparator 1524 compares the sensed temperature sensed by the sensor 1502 to a pre-determined threshold to determine whether or not the sensed temperature inside the insulin infusion pump case 1550, or of the insulin vial 1556, exceeds the pre-determined threshold. Typically the threshold temperature is set to 80 degrees Fahrenheit.

Indicator 1534 is disposed on the case 1550 such that at least a portion of the indicator 1534 is visible from outside the case 1550. The indicator 1534 may display an illuminated light, provide a buzzer alert audible sound, provide a multi-colored illuminated alert or provide a combination of illumination of colors and/or sounds.

The display module 1560 includes display screen 1562 and display buttons 1564. The display module 1560 is operatively coupled to microprocessor 1530 and is used to display information on display screen 1562. A user may modify the information displayed on screen 1562 by manipulating buttons 1564, which control the format and/or content of information displayed on display screen 1562.

As described in relation to FIG. 6, the insulin cases 1450 and 1550 may be wirelessly connected to one or more remote devices, such as smartphone, tablet, PC, or any other device capable of wireless communication.

It will be appreciated from the above that the embodiments may be implemented as computer software, which may be supplied on a storage medium or via a transmission medium such as a local-area network or a wide-area network, such as the Internet. It is to be further understood that, because some of the constituent system components and method steps depicted in the accompanying figures can be implemented in software, the actual connections between the systems components (or the process steps) may differ depending upon the manner in which the present invention is programmed. Given the teachings provided herein, one of ordinary skill in the related art will be able to contemplate these and similar implementations or configurations.

It is to be understood that the above-described can be implemented in various forms of hardware, software, firmware, special purpose processes, or a combination thereof. Some embodiments can be implemented in software as an application program tangible embodied on a computer readable program storage device. The application program can be uploaded to, and executed by, a machine comprising any suitable architecture.

The particular embodiments disclosed above are illustrative only, and may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit. Although illustrative embodiments have been described in detail herein with reference to the accompanying drawings, it is to be understood that the disclosure is not limited to those precise embodiments, and that various changes and modifications can be effected therein by one skilled in the art without departing from the scope and spirit of the invention as defined by the appended claims.

Unless otherwise defined, all technical and scientific terms used herein have the same meaning as commonly understood by one of ordinary skill in the art to which this invention belongs. Although methods and materials similar to or equivalent to those described herein can be used in the practice or testing of the monitoring system, suitable methods and materials are described above. All publications, patent applications, patents, and other references mentioned herein are incorporated by reference in their entirety to the extent allowed by applicable law and regulations. The monitoring system may be embodied in other specific forms without departing from the spirit or essential attributes thereof, and it is therefore desired that the present embodiment be considered in all respects as illustrative and not restrictive. Any headings utilized within the description are for convenience only and have no legal or limiting effect.

What is claimed is:

1. A monitoring system for monitoring parameters of an insulin storage case comprising:
a case structure adapted to contain a sample of insulin;
a temperature sensor disposed in the interior of the case that senses a temperature of the interior of the case;
a comparator disposed in the interior of the case that receives input from the temperature sensor indicative of the temperature of the interior of the case and generates an output signal as a function of the sensed temperature of the interior of the case; and
an indicator disposed on the case such that at least a portion of the indicator is visible from the exterior of the case, the indicator operatively coupled to the comparator, wherein the indicator provides an indication of the temperature of the interior of the case based on the output signal generated by the comparator of the sample.

2. The monitoring system as claimed in claim 1, wherein the case is an infusion pump case.

3. The monitoring system of claim 1, further comprising a bi-directional communication link adapted to transmit a representation of the indication of the sample.

4. The monitoring system of claim 3, wherein the bi-directional communication link is a wireless link.

5. The monitoring system of claim 1, wherein the comparator generates a warning indication based on a temperature profile of the sample, and wherein the indicator is adapted to provide an indication representative of the warning indication.

6. The apparatus of claim 5, wherein the comparator generates a fail indication based on the temperature profile of the sample, and wherein the indicator is adapted to provide an indication representative of the fail indication.

7. The apparatus of claim 1, further comprising an ultra-violet sensor, operatively coupled to the comparator, to provide a signal to the comparator based on sensed ultra-violet exposure of the sample.

8. The apparatus of claim 1, further comprising a pressure sensor, operatively coupled to the comparator, to provide a signal to the comparator based on sensed pressure conditions experienced by the sample.

9. The apparatus of claim 1, further comprising a plurality of indicators, each indicator operatively coupled to the comparator, wherein each indicator is associated with a particular sensed parameter of the sample.

10. The apparatus of claim 9, wherein each indicator is a visual indicator and provides a color output based on the output generated by the comparator.

11. The apparatus of claim 1, wherein the indication provided by the indicator is an audible indication.

12. The apparatus of claim 11, wherein the audible indication is a tone that is a function of the output signal generated by the comparator.

13. The apparatus of claim 1, further comprising a plurality of sensors, each sensor operatively coupled to the comparator and each sensor adapted to sense an associated parameter of the interior of the case and provide a sensed signal to the comparator as a function of a particular parameter of the associated sensor.

14. A method for monitoring a temperature of an insulin storage case comprising:
sensing a temperature of the interior of the insulin case;
generating an output signal as a function of the sensed temperature of the interior of the case; and
providing an indication of the temperature of the interior of the case based on the output signal generated.

15. The monitoring method as claimed in claim 14 wherein the case is an infusion pump case.

16. The method of claim 14, further comprising transmitting a representation of a profile of the sample.

17. The method of claim 16, wherein the transmitting is a wireless transmission.

18. The method of claim 14, wherein the generating step generates a warning indication based on a profile of the sample, and the providing step provides an indication representative of the warning indication.

19. The method of claim 18, wherein the generating step generates a fail indication based on the profile of the sample, and the providing step provides an indication representative of the fail indication.

20. An insulin temperature sensing system that senses temperature of a sample of insulin comprising:
a housing having an interior portion and an exterior portion, the housing comprising:
a sensor that senses a temperature of the interior portion of the housing and generates a sensed temperature signal;
a processor, operatively coupled to the sensor, the processor receives the sensed temperature signal and generates an output signal when the received sensed temperature signal indicates that the temperature of the interior of the housing exceeds a pre-determined temperature, wherein the output signal generated by the processor is indicative of the magnitude that the sensed temperature has exceeded the pre-determined threshold temperature; and
an indicator, operatively coupled to the processor that receives the output signal from the indicator and provides a visible indication of the output signal, wherein the visible indication is a color indication that provides an indication of the magnitude that the sensed temperature of the interior of the housing exceeded the pre-determined threshold temperature; and
an antenna, disposed on the housing, operatively coupled to the processor, to transmit a wireless signal, via a wireless network, from the housing to a remote device, the wireless signal based on the output signal generated by the processor.
